(19) United States
(12) Patent Application Publication
(10) Pub. No.: US 2013/0274818 A1
(43) Pub. Date: Oct. 17, 2013

(54) CANNULATED LOCKING BONE FASTENER

(71) Applicants: Mojan Goshayeshgar, Atherton, CA (US); Amy Arthur, Mountain View, CA (US)

(72) Inventors: Mojan Goshayeshgar, Atherton, CA (US); Amy Arthur, Mountain View, CA (US)

(73) Assignee: KYPHON SARL, Neuchatel (CH)

(21) Appl. No.: 13/446,347

(22) Filed: Apr. 13, 2012

(51) Int. Cl. A61B 17/86

(52) U.S. Cl. 606/310

(57) ABSTRACT

A bone fastener system including a bone fastener having cavities for receiving locking screws traverse to a longitudinal axis. The bone fastener includes a proximal end connected to a head, a distal end and an elongated shank defining a longitudinal axis. The elongated shank has an inner surface defining an inner cavity extending from the proximal end to the distal end along the longitudinal axis. The inner cavity is configured to receive a guide wire. The system includes a first locking screw and the bone fastener further comprises a second cavity defined by an inner surface disposed at an angle transverse to the longitudinal axis configured to receive a first locking screw.

### TECHNICAL FIELD

The present disclosure generally relates to medical devices for the treatment of bone disorders, and more particularly to a cannulated locking bone fastener configured to receive locking screws to prevent the bone fastener from shifting.

### BACKGROUND

Many surgical orthopedic procedures require screws specifically designed for use in bone tissue. These surgical procedures are often performed to correct a fractured bone or vertebrae caused by disease, trauma, or both. A wide array of bone screws exist which are adapted to perform specific functions or to be compatible with a specific type of bone tissue, procedure and/or orthopedic implant being used in the procedure.

For example, in order to stay a bone fracture, particularly in a long bone, a surgeon may insert a bone fracture reduction rod, or intramedullary nail, into an intramedullary canal of the bone. In order to secure the rod, the surgeon will use multiple bone screws. Screws used for this purpose often extend through the bone and either engage the implant or span the length of the entire fracture. The use of such screws provides many advantages such as increasing the rotational stability of the implant enhancing the union rate of the bone, and promoting limb rehabilitation but often are too rigid when locked in place. This results in relative lack of motion at the fracture site, and can in some situations, be too stiff to allow fracture healing.

Accordingly, what is needed is an orthopedic screw system that increases the rotational stability of an engaged implant, enhances the union rate of the bone, promotes limb rehabilitation and provides ample stabilization without being too stiff. This disclosure describes an improved fenestrated bone fastener that provides for improved anchorage in bone over the prior art technologies.

### SUMMARY

Accordingly, a bone fastener system for providing stabilization of fractures without being too stiff for articulation and healing is described. The bone fastener system in accordance with the present disclosure can be used to treat fractures and in particular to treat simple articular and metaphyseal fractures.

In one particular embodiment, in accordance with the principles of the present disclosure, a bone fastener system comprises a bone fastener having cavities for receiving locking screws traverse to a longitudinal axis. The bone fastener includes a proximal end connected to a head, a distal end and an elongated shank defining a longitudinal axis. The elongated shank includes an inner surface that defines an inner cavity extending from the proximal end to the distal end along the longitudinal axis. The inner cavity is configured to receive a guide wire. The system includes a first locking screw. The bone fastener includes a second cavity defined by an inner surface disposed at an angle transverse to the longitudinal axis configured to receive the first locking screw.

In one particular embodiment, in accordance with the principles of the present disclosure, a bone fastener system comprises a bone fastener having cavities for receiving locking screws traverse to a longitudinal axis. The bone fastener includes a proximal end connected to a head, a distal end and an elongated shank defining a longitudinal axis. The elongated shank includes an inner surface that defines an inner cavity extending from the proximal end to the distal end along the longitudinal axis. The inner cavity is configured to receive a guide wire. The system includes a first locking screw. The bone fastener includes a second cavity defined by an inner surface disposed at an angle transverse to the longitudinal axis configured to receive the first locking screw. The system includes a second locking screw. The bone fastener includes a third cavity defined by an inner surface disposed at an angle transverse to the longitudinal axis configured to receive the second locking screw.

In another embodiment of the present disclosure, a method of securing a bone fastener to bone using the bone fastener system of present disclosure is provided. The method comprises threading the bone fastener into bone across at least a portion of a bone defect to be treated. A guide wire is set in place in order to guide the bone fastener into the proper location. Once the bone fastener is set in place, a first locking screw is threaded into the second inner cavity disposed at an angle transverse to the longitudinal axis so as to lock the bone fastened into position thereby stabilizing the bone defect. This allows for stabilization of the bone fastener without compromising articulation that is necessary for healing.

In one particular embodiment, in accordance with the principles of the present disclosure, a locking bone fastener kit is provided. The kit comprises at least one bone fastener in accordance with the bone fastener system of the present disclosure. The bone fastener includes a distal end and a proximal end wherein the proximal end is attached to a head and the distal end is self-tapping or not self-tapping. The bone fastener also includes an elongated shank defining a longitudinal axis having an inner surface configured to define a longitudinal cavity. The longitudinal cavity extends along the longitudinal axis of the elongated shank and is configured to receive a guide wire. The elongated shank of the bone fastener further comprises at least one additional cavity defined by an inner surface that is transverse to the longitudinal axis and is configured to receive a locking screw. The inner surface can include a thread form that spans the entire transverse cavity, part of the transverse cavity or has no thread form at all. The kit further comprises at least one locking screw configured to fit or thread into the at least one transverse cavity so as to stabilize the bone fastener in place without being overly stiff thereby facilitating articulation of the fractured area. This leads to better and more complete healing of the bone fracture or defect.

The bone fastener system and the method of the present disclosure is further described in the figures and following sections.

### BRIEF DESCRIPTION OF THE DRAWINGS

The present disclosure will become more readily apparent from the specific description accompanied by the following drawings, in which:

FIG. 1 is a side view of one particular embodiment of a bone fastener system in accordance with the principles of the present disclosure;

FIG. 2 is a cross sectional view of the components of the bone fastener system of FIG. 1;

FIG. 3 is a side view of one particular embodiment of a bone fastener system in accordance with the principles of the present disclosure;

FIGS. 4a-4c are cross sections of the components of FIG. 3; and

FIG. 5 is a side perspective view of the bone fastener system of FIG. 3 disposed in bone.

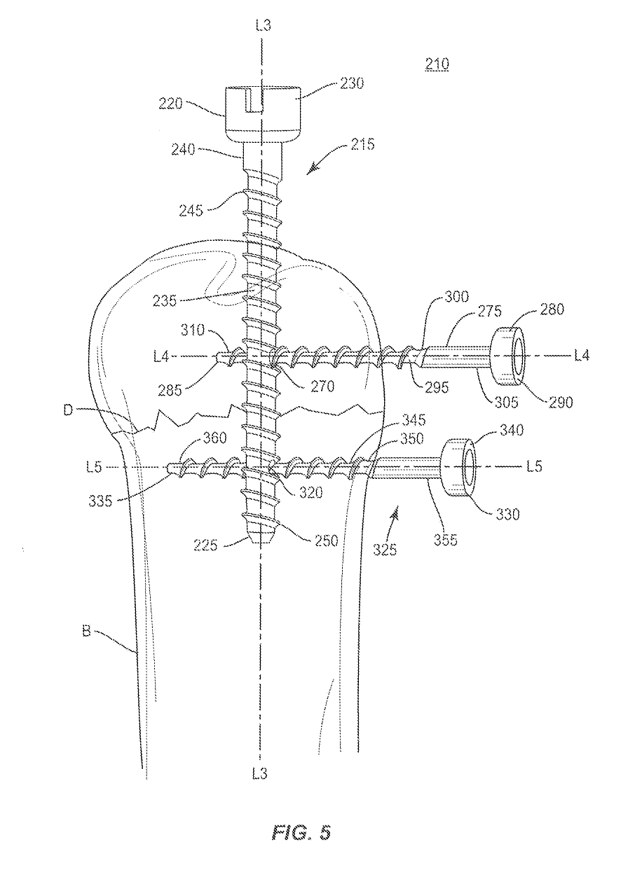

Like reference numerals indicate similar parts throughout the figures.

### DETAILED DESCRIPTION OF THE INVENTION

The exemplary embodiments of the bone fastener and methods of use disclosed are discussed in terms of medical devices for the treatment of bone disorders and more particularly, in terms of a fenestrated bone fastener having an elongated cannulated core and at least one cavity configured to receive a locking screw. The bone fastener may also be sprayed, layered, fused, coated or textured in a manner or with material that facilitates the growth and attachment of bone. The locking screw bone fastener improves the initial stability of the bone fastener in dynamic fixation devices. It is envisioned that employment of the coated bone fastener of the present disclosure can be used with other implants, for example, such as a vertebral rod system that provides stability in dynamic fixation devices. The coated bone fastener may also be used with other constructs such as plates.

It is envisioned that the fenestrated bone fastener of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures, of such disorders, such as, for example, discectomy, laminectomy, fusion, bone graft, implantable prosthetics and/or dynamic stabilization applications. It is contemplated that the present disclosure may be employed with other osteal and bone related applications, including those associated with diagnostics and therapeutics. It is further contemplated that the disclosed fenestrated bone fastener may be employed in a surgical treatment with a patient in a prone or supine position, employing a posterior, lateral or anterior approach.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

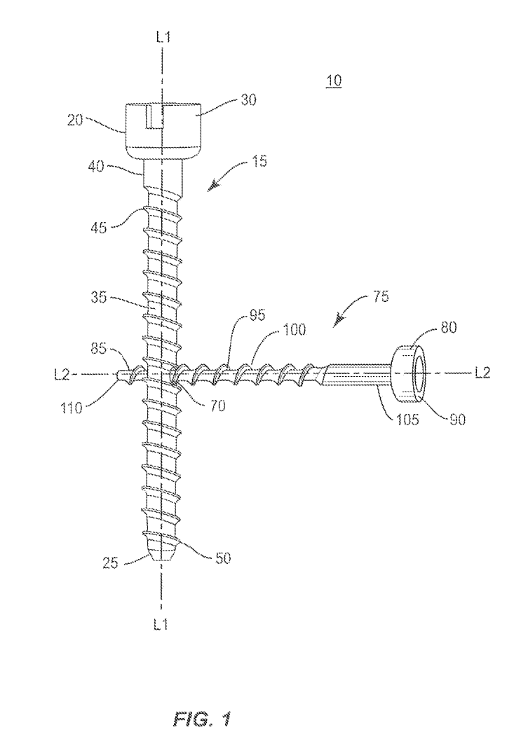

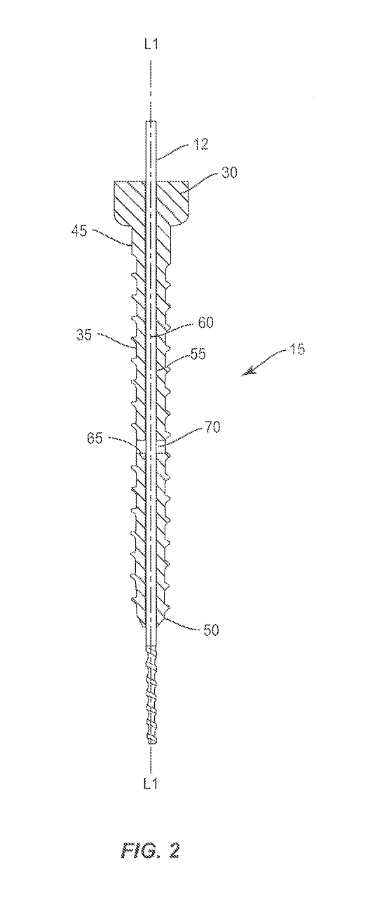

The following discussion includes a description of a fenestrated bone fastener, related components and exemplary methods of employing the bone fastener in accordance with the principles of the present disclosure. Alternate embodiments are also disclosed. Reference will now be made in detail to the exemplary embodiments of the present disclosure, which are illustrated in the accompanying figures. Turning now to FIGS. 1-2, there is illustrated components of a fenestrated bone fastener system 10 in accordance with the principles of the present disclosure.

Bone fastener 15 extends between a proximal end 20 and a distal end 25. Proximal end 20 includes a head 30 configured for engagement with a tool such as, for example, a driver, not shown. Distal end 25 is configured with cutting features so as to facilitate cutting into bone as it is inserted. An elongated section, such as, for example, an elongated shank 35 extends from head 30 to distal end 25. Shank 35 includes an outer surface 40, a proximal end 45 and a distal end 50. It is contemplated that shank 35 of bone fastener 15 can be variously dimensioned, for example, with regard to the length or thickness, and cross sectional geometry such as those discussed above. For example, the cross-sectional geometries of shank 35 of the bone fastener defining the outer surface and/or inner surface can be round, oval, rectangular, irregular, consistent, variable, uniform and non-uniform, and the inner and outer surfaces may have the same or different cross section geometry. That is, the outer and inner surfaces can be concave, convex, flat, arcuate, multifaceted, or a combination thereof. Shank 35 is threaded along the length thereof and configured for penetrating tissue. Shank 35 has a cylindrical cross section configuration and includes an outer surface having an external thread form. It is contemplated that the thread form may include a single thread turn or a plurality of discrete threads. It is further contemplated that other engaging structures may be located on shank 35, such as, for example, a nail configuration, barbs, expanding elements, raised elements and/or spikes to facilitate engagement of shank 35 with tissue.

Proximal end 45 of shank 35 is connected to head 30 which can be used to drive bone fastener 15 into bone or can be configured to support a bone construct. Head 30 can be made from the same or different material than shank 35 and can be either connected to shank 35 or formed as a monolithic unit. Head 30 can be attached to the shank 35 so that it can rotate and is angled, is positionable, or fixed. Head 30 can also have different configurations depending on the particular use of the fenestrated bone fastener 15.

As shown in FIG. 2, shank 35 has a body that extends from proximal end 45 to distal end 50. It is contemplated that shank 35 or only portions thereof can have variously dimensioned, for example, with regard to length, width, diameter and thickness. It is further contemplated that in addition to a cylindrical cross-sectional geometry, shank 35 or only portions thereof, can have various configurations, for example, round, oval, rectangular, irregular, consistent, variable, uniform and non-uniform.

Shank 35 includes a first inner surface 55 defining an elongated cavity 60, such as a cannulated configuration, as shown in FIG. 2. Elongated cavity 60 extends partially or continuously from proximal end 45 to distal end 50 of shank 35 along a longitudinal axis L1. Elongated cavity 60 is configured to receive a guide wire 12. It is contemplated that inner surface 55 be continuous, non-interrupted, smooth, textured, or have a porous configuration and is disposed in close fitting engagement with the outer surface that forms the shape and contour of the shank 35.

Shank 35 includes a second inner surface 65 defining a second cavity 70 disposed transversely to first inner surface 55. Cavity 70 is configured for disposal of a locking screw 75, (shown in FIG. 1), discussed herein. Second cavity 70 extends either partially or completely through the shank 35. It is contemplated that second cavity 70 has different configurations, for example, such as spherical, oval, rectangular, triangular, elliptical, and polygonal. First inner surface 55 can be threaded, partly threaded or non-threaded.

System 10 shown in FIG. 1, includes a locking screw 75 for disposal and/or engagement with second cavity 70. Locking screw 75 extends between a proximal end 80 and a distal end 85 along an axis L2. Proximal end 80 includes a head 90 configured for engagement with a tool such as, for example, a driver, not shown. An elongated section, such as, for example, an elongated shank 95 extends from head 90 to distal end 85. Shank 95 includes an outer surface 100, a proximal end 105 and a distal end 110. It is contemplated that shank 95 or only portions thereof can have variously dimensioned, for example, with regard to length, width, diameter and thickness.

It is further contemplated that shank 95 or only portions thereof, can have various configurations, for example, round, oval, rectangular, irregular, consistent, variable, uniform and non-uniform. Shank 95 is threaded along the length thereof and configured for penetrating tissue. Shank 95 has a cylindrical cross section configuration and includes an outer surface having an external thread form. It is contemplated that the thread form may include a single thread turn or a plurality of discrete threads. It is further contemplated that other engaging structures may be located on shank 95, such as, for example, a nail configuration, barbs, expanding elements, raised elements and/or spikes to facilitate engagement of shank 95 with tissue.

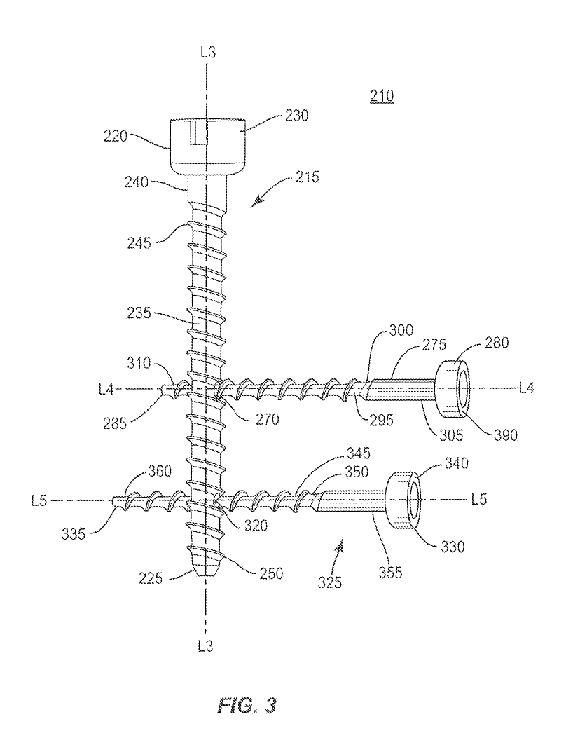

In another embodiment, shown in FIG. 3, system 210 includes a bone fastener 215 extending between a proximal end 220 and a distal end 225. Proximal end 220 includes a head 230 configured for engagement with a tool such as, for example, a driver, not shown. An elongated shank 235 extends from head 230 to distal end 225. Shank 235 includes an outer surface 240, a proximal end 245 and a distal end 250. Proximal end 245 of shank 235 is connected to head 230 which can be used to drive bone fastener 215 into bone or can be configured to support a bone construct.

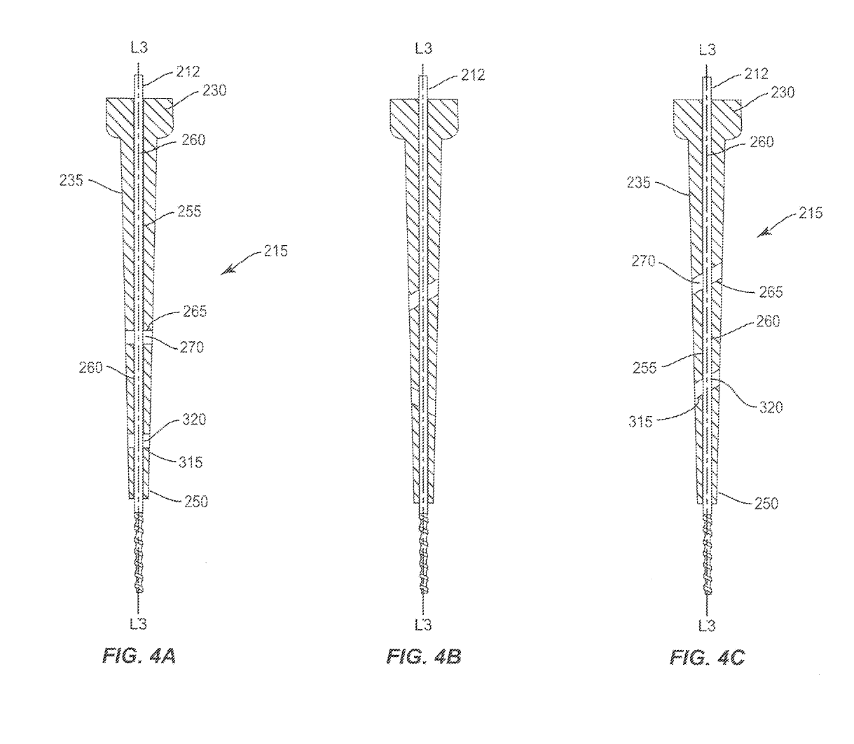

Shank 235 has a body that extends from proximal end 245 to distal end 250. Shank 235 includes a first inner surface 255 defining an elongated cavity 260, such as a cannulated configuration, as shown in FIGS. 4a-4c. Elongated cavity 260 extends partially or continuously from proximal end 45 to distal end 250 of shank 235 along a longitudinal axis L3.

Shank 235 includes a second inner surface 265 defining a second cavity 270 disposed transversely to first inner surface 255 for disposal of a locking screw. Locking screw 275 is configured for disposal in second cavity 270. Locking screw 275 extends between a proximal end 280 and a distal end 285 along an axis L4. Proximal end 280 includes a head 290 configured for engagement with a tool such as, for example, a driver, not shown. An elongated section, such as, for example, an elongated shank 295 extends from head 290 to distal end 285. Shank 295 includes an outer surface 300, a proximal end 305 and a distal end 310.

Shank 235 includes a third inner surface 315 defining a third cavity 320 disposed transversely to first inner surface 355. Third cavity 320 extends either partially or completely through the shank 335. It is contemplated that third cavity 320 has different configurations, for example, such as spherical, oval, rectangular, triangular, elliptical, and polygonal. Third inner surface 315 can be threaded, partly threaded or non-threaded.

It is contemplated that second and third cavities, 270 and 320, can have different spatial orientations, for example, such as angular, off-centered to one another, evenly spaced apart form one another, and unevenly spaced apart from one another. An angular orientation of cavities 270 and 320 with respect to each other can span between an angle of about 10 to about 120 degrees. Longitudinal cavity 260 formed from first inner surface 255 is in communication with second and third cavities 270, 320.

A second locking screw 325 is configured for disposal in third cavity 320. Locking screw 320 extends between a proximal end 330 and a distal end 335 along an axis L5. Proximal end 330 includes a head 340 configured for engagement with a tool. An elongated section, such as, for example, an elongated shank 345 extends from head 340 to distal end 335. Shank 345 includes an outer surface 350, a proximal end 355 and a distal end 360. It is contemplated that shank 345 or only portions thereof can have variously dimensioned, for example, with regard to length, width, diameter and thickness. Shank 246 is threaded along the length thereof and configured for penetrating tissue. Shank 345 has a cylindrical cross section configuration and includes an outer surface having an external thread form. It is contemplated that the thread form may include a single thread turn or a plurality of discrete threads. It is further contemplated that other engaging structures may be located on shank 345, such as, for example, a nail configuration, barbs, expanding elements, raised elements and/or spikes to facilitate engagement of shank 345 with tissue.

In assembly, operation and use, the bone fastener 215 is employed with a surgical procedure for treatment of a bone defect affecting a bone of a patient, as discussed herein. That is, bone fastener 215 is used as part of surgical procedures for treatment of a condition or injury of an affected section D of the bone B. A guide wire 212 is inserted into the bone across at least a portion of the bone defect. Guide wire 212 is inserted through cavity 260 along longitudinal axis L3 to guide bone fastener 215 to the proper fixation location. Bone fastener 215 can be threaded or tapped into place once positioned. Once bone fastener is properly positioned, guide wire 212 is pulled out of bone fastener 215 and out of the body. Locking screw 275 is inserted in a transverse orientation to bone fastener 215 into cavity 270 and positioned to prevent bone fastener 215 from dislodging or moving out of place. Locking screw 325 is inserted in a transverse orientation to bone fastener 215 into cavity 320 and positioned to prevent bone fastener 215 from dislodging or moving out of place.

In use, to treat the affected section of the bone, a medical practitioner obtains access to a surgical site in any appropriate manner, such as through incision and retraction of tissues. It is envisioned that the bone fastener 215 may be used in any existing surgical method or technique including open surgery, mini-open surgery, minimally invasive surgery and percutaneous surgical implantation, whereby bone B is accessed through a micro-incision, or sleeve that provides a protected passageway to the area. Once access to the surgical site is obtained, the particular surgical procedure is performed for treating the bone injury.

Bone fastener 215 may be employed as a bone screw, pedicle screw or multi-axial screw used in surgery. It is contemplated that the all or part of the outer surfaces 240 and 210, the inner surfaces defining traverse cavities 270, 320 or the elongated cavity 260 may be coated, sprayed, fused, or textured with an osteoconductive and/or osteoinductive material in a manner or with material that facilitates the growth and attachment of bone. The osteoconductive and/or osteoinductive material used is selected from the group consisting of is selected from the group consisting of bone graft, therapeutic polynucleotides or polypeptides, rigid polymers, biocompatible metals, such as titanium elements, metal powders of titanium or titanium compositions, sterile bone materials, such as allograft or xenograft materials, synthetic bone materials such as coral and calcium compositions, such as hyaluronic acid (HA), calcium phosphate and calcium sulfite, biologically active agents, such as, Bone Morphogenetic Proteins (BMP), Growth and Differentiation Factors Proteins (GDF), and cytokines, allogenic demineralized bone, synthetic polymers and copolymers, synthetic copolymers of polyglicolic and polylactic acid, purified collagen, epidermal growth factor (EGF) platelet derived growth factor (PDGF), fibroblast growth factors (FGFs), parathyroid hormone related peptide (PTHrp), insulin-like growth factors (IGFs) and transforming growth factor-beta (TGF-B).

In one embodiment, in accordance with the principles of the present disclosure, first inner surface 255 of the bone fastener 215 defining the elongated cavity 260 and the traverse cavities 270, 320 are coated, sprayed, fused, or textured with an osteoconductive and/or osteoinductive material and the outer surface of the bone fastener is not. This directs growth into the fenestrations and the elongated cavity of the fenestrated bone fastener that may be beneficial depending on where the bone fastener is being used. The first inner surface 255 of the bone fastener 215 defining the elongated cavity 260 and the traverse cavities 270, 320 can be coated, sprayed, fused, or textured with the same, different or a combination of materials with osteoconductive and/or osteoinductive material selected from the group consisting of is selected from the group consisting of bone graft, therapeutic polynucleotides or polypeptides, rigid polymers, biocompatible metals, such as titanium elements, metal powders of titanium or titanium compositions, sterile bone materials, such as allograft or xenograft materials, synthetic bone materials such as coral and calcium compositions, such as hyaluronic acid (HA), calcium phosphate, calcium sulfite, biologically active agents, such as, Bone Morphogenetic Proteins (BMP), Growth and Differentiation Factors Proteins (GDF), cytokines, allogenic demineralized bone, synthetic polymers and copolymers, synthetic copolymers of polyglicolic and polylactic acid, purified collagen, epidermal growth factor (EGF) platelet derived growth factor (PDGF), fibroblast growth factors (FGFs), parathyroid hormone related peptide (PTHrp), insulin-like growth factors (IGFs) and transforming growth factor-beta (TGF-B).

In another embodiment, in accordance with the principles of the present disclosure, the outer surface 240 of the shank 235 along with first inner surface 255 of the bone fastener 215 defining the elongated cavity 260 and the traverse cavities 270, 320 and not the elongated cavity 235 can be coated, sprayed, fused, or textured with an osteoconductive and/or osteoinductive material in a manner or with material that facilitates the growth and attachment of bone, for example with an osteoconductive and/or osteoinductive material selected from the group consisting of is selected from the group consisting of bone graft, therapeutic polynucleotides or polypeptides, rigid polymers, biocompatible metals, such as titanium elements, metal powders of titanium or titanium compositions, sterile bone materials, such as allograft or xenograft materials, synthetic bone materials such as coral and calcium compositions, such as hyaluronic acid (HA), calcium phosphate and calcium sulfite, biologically active agents, such as, Bone Morphogenetic Proteins (BMP), Growth and Differentiation Factors Proteins (GDF), and cytokines, allogenic demineralized bone, synthetic polymers and copolymers, synthetic copolymers of polyglicolic and polylactic acid, purified collagen, epidermal growth factor (EGF) platelet derived growth factor (PDGF), fibroblast growth factors (FGFs), parathyroid hormone related peptide (PTHrp), insulin-like growth factors (IGFs) and transforming growth factor-beta (TGF-B).

The outer and inner surfaces can be partially coated or completely coated in order to enhance bony fixation. The outer surface can also be textured so as to enhance bone growth about the bone fastener. As mentioned above, the same osteoconductive and/or osteoinductive materials can be used to coat each surface or in the alternative, a mixture of at least two different osteoconductive and/or osteoinductive materials can be used to coat all or part of the inner and outer surfaces. Bone mineral matrix, allograft, autograft and other materials that facilitate initial bone growth can be inserted inside the elongated and/or transverse cavities 70, 80 of the bone fastener just before implantation in order to provide better initial bone growth within the elongated and/or traverse cavities 70, 80.

In one embodiment, in accordance with the principles of the present disclosure bone fastener 215 can be pre-packed with Bone mineral matrix, allograft, autograft and/or other materials that facilitate initial bone growth. For example, in one embodiment of the present disclosure, autograft material is loaded inside the elongated cavity 235 and/or the traverse cavities 270, 320. This pre-packing feature allows the bone fastener 215 to be coated, sprayed, fused, or textured with an osteoconductive and/or osteoinductive material in a manner or with material that facilitates the growth and attachment and to be loaded with Bone Matrix material so as to improve biological fixation of the bone fastener when used.

The components of bone fastener 215 are fabricated from materials suitable for medical applications, including metals, polymers, ceramics, biocompatible materials and/or their composites, depending on the particular application and/or preference of a medical practitioner. For example, bone fastener 05, discussed below, can be fabricated from materials such as commercially pure titanium, titanium alloys, Grade 5 titanium, super-elastic titanium alloys, cobalt-chrome alloys, stainless steel alloys, superelastic metallic alloys (e.g. Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof such as calcium phosphate (e.g. SKELITE™ manufactured by Biologix Inc.), rigid polymers including polyphenylene, polyamide, polyimide, polyetherimide, polyethylene, polyurethanes of any durometer, epoxy and silicone. Different components of the bone fastener may have alternative material composites to achieve various desired characteristics such as strength, rigidity, elasticity, compliance, biomechanical performance, durability and radiolucency or imaging preference. The components of the bone fastener may also be fabricated from a heterogeneous material such as a combination of two or more of the above-described materials.

It is envisioned that the components of the system can be manufactured via various methods. For example, the components can be manufactured and assembled via injection-molding, insert-molding, overmolding, compression molding, transfer molding, co-extrusion, pultrusion, dip-coating, spray-coating, powder-coating, porous-coating, machining, milling from a solid stock material, and their combinations. One skilled in the art, however, will realize that such materials and fabrication methods suitable for assembly and manufacture, in accordance with the present disclosure, would be appropriate.

Bone fastener 215 can be employed alone or with other hardware, which are configured for attachment to bone during surgical treatment of a bone defect.

Radiomarkers may be included in the coating of the outer and/or inner surfaces of the bone fastener 215 for identification under x-ray, fluoroscopy, CT or other imaging techniques. Metallic or ceramic radiomarkers, such as tantalum beads, tantalum pins, titanium pins, titanium endcaps and platinum wires can be used as part of the bone fastener 215 or in conjunction with the bone fastener 15.

A bone fixation kit is also provided. The kit includes at least one guide wire, bone fastener and at least one locking screw, in accordance with the present disclosure. The kit includes at least one tool configured to engage the head of the bone fastener and the at least one locking screw to drive it into position and an alignment mechanism.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A bone fastener system comprising:
a bone fastener comprising an elongated shank defining a longitudinal axis having a proximal end and a distal end wherein the proximal end is connected to a head, the elongated shank comprising an inner surface defining an inner cavity extending from the proximal end to the distal end along the longitudinal axis and is configured to receive a guide wire;
a first locking screw; and
a second inner surface disposed at an angle transverse to the longitudinal axis in the bone fastener defining a second cavity configured to receive the first locking screw.

2. A bone fastener system of claim 1 wherein at least a portion of the inner surface of the second cavity comprises a thread form configured to secure the first locking screw to the bone fastener.

3. A bone fastener system of claim 1 further comprising a third cavity defined by an inner surface in the bone fastener disposed at an angle transverse to the longitudinal axis that is configured to receive a second locking screw and the bone fastener.

4. A bone fastener system of claim 3 wherein at least a portion of the inner surface defining the first cavity and the inner surface defining the second cavity comprises a thread form configured to secure the first and second locking screws to the bone fastener and the bone fastener to bone.

5. A bone fastener system according to claim 1 wherein an outer surface of the bone fastener contains threads that are configured to thread into bone and the distal end of the elongated shank is configured so as to be self-tapping.

6. A bone fastener system according to claim 1 wherein an outer surface defining the elongated shank of the bone fastener further comprises bone conductive material disposed thereon so as to promote bone growth to further secure the bone fastener to bone.

7. A bone fastener system according to claim 3 wherein the second and third inner cavities are disposed at different angles with respect to each other.

8. A bone fastener system according to claim 1 wherein the distal end of the bone fastener comprises cutting features configured to cut into bone as the bone fastener is inserted into bone.

9. A bone fastener system according to claim 5, wherein the bone conductive material is sprayed, layered, fused, coated or textured in a manner or with material that facilitates the growth and attachment of bone and the bone conductive material is selected from the group consisting of bone graft, therapeutic polynucleotides or polypeptides, rigid polymers, biocompatible metals, such as titanium elements, metal powders of titanium or titanium compositions, sterile bone materials, such as allograft or xenograft materials, synthetic bone materials such as coral and calcium compositions, such as hyaluronic acid (HA), calcium phosphate, calcium sulfite, biologically active agents, such as, Bone Morphogenetic Proteins (BMP), Growth and Differentiation Factors Proteins (GDF), cytokines, allogenic demineralized bone, synthetic polymers and copolymers, synthetic copolymers of polyglicolic and polylactic acid, purified collagen, epidermal growth factor (EGF) platelet derived growth factor (PDGF), fibroblast growth factors (FGFs), parathyroid hormone related peptide (PTHrp), insulin-like growth factors (IGFs) and transforming growth factor-beta (TGF-B).

10. A method of securing a bone fastener to bone comprising providing the bone fastener system of claim 1;
threading the bone fastener into bone across at least a portion of a bone defect to be treated; and
threading a first locking screw into the second inner cavity disposed at an angle transverse to the longitudinal axis so as to lock the bone fastened into position thereby stabilizing the bone defect.

11. A method of claim 10 wherein the bone fastener comprises a third cavity disposed at an angle transverse to the longitudinal axis, wherein the method further comprises threading a second locking screw into the third cavity so as to further secure the bone fastener to the bone.

12. A method of claim 10 further comprising:
positioning a guide wire in the bone prior to threading the bone fastener into bone;
positioning the bone fastener over the guide wire; and
threading the bone fastener into bone across the bone defect.

13. A method of claim 12 further comprising removing the guide wire and threading the first locking screw into the second cavity so as to lock the bone fastener into position.

14. A method of claim 13 further comprising threading a second locking screw into the third cavity disposed at an angle transverse to the longitudinal axis so as to further secure the bone fastener to bone.

15. A locking bone fastener kit comprising:
at least one bone fastener having a distal end and a proximal end wherein the proximal end is attached to the head, the bone fastener comprising an elongated shank defining a longitudinal axis having an inner surface defining a longitudinal cavity configured to receive a guide wire and at least one additional cavity transverse to the longitudinal axis configured to receive a locking screw; and
at least one locking screw configured to fit into the at least one additional cavity transverse to the longitudinal axis.

16. A locking bone fastener kit of claim 15 further comprising at least one guide wire configured to fit into the longitudinal cavity of the shank so as to guide the bone fastener into a desired position in bone.

17. A locking bone fastener kit of claim 15 further comprising at least one tool configured to engage the head of the bone fastener and the at least one locking screw to drive it into position and an alignment mechanism.

18. A locking bone fastener kit of claim 15 wherein at least one of the bone fastener and at least one locking screw comprises self-taping tip.

19. A locking bone fastener kit of claim 15 further comprising bone cement, bone conductive material, bone inductive material, bone graft materials, therapeutic polynucleotides or polypeptides, allograft or xenograft materials, synthetic bone materials, biologically active agents, Bone Morphogenic Proteins (BMP) Growth and Differentiation Factors Proteins (GDF), allogenic demineralized bone, hyaluronic acid (AA), calcium phosphate, calcium sulfite and combinations thereof.

20. A locking bone fastener system comprising:
at least one bone fastener comprising a head and an inner surface defining a longitudinal cavity configured to receive a guide wire, the bone fastener having a proximal and distal end wherein the proximal end is connected to the head and the distal end is self-tapping;
the elongated shank having a first inner surface defining a threaded transverse cavity and a second inner surface defining a second threaded transverse cavity; and
a first and a second locking screw configured to thread into the first and second threaded transverse cavities so as to secure the bone fastener into bone.
